(19) United States
(12) Patent Application Publication
(10) Pub. No.: US 2007/0100147 A1
(43) Pub. Date: May 3, 2007

(54) Process for preparing raloxifene hydrochloride

(76) Inventors: Massimo Ferrari, Cenate Sotto (IT); Fabrizio Zinetti, Casazza (IT); Paolo Belotti, San Paolo D'Argon (IT)

Correspondence Address: Jay S Cinamon;Abelman Frayne & Schwab, 10th Floor, 666 Third Avenue, New York, NY, 10017, US

(21) Appl. No.: 10/562,762

(22) PCT Filed: Jun. 28, 2004
(86) PCT No.: PCT/EP04/51263

§ 371 Date: Dec. 27, 2005

(30) Foreign Application Priority Data
Jun. 30, 2003 (IT) MI2003A001333

(51) Int. Cl. A61K 31/453; C07D 409/02

(52) U.S. Cl. 546/197

(57) ABSTRACT

Process for preparing raloxifene hydrochloride with a purity greater than 98% and low aluminium content comprising the following stages a) demethylation of 6-methoxy-2-(4-methoxyphenyl)benzo[b]thiophene in pyridine and hydrochloric acid to obtain 6-hydroxy2-(4-hydroxyphenyl)benzo[b]thiophene in pyridine hydrochloride, b) acetylation of 6-hydroxy-2-(4hydroxyphonyl)benzo[b]thiophene with an acetylating agent to obtain the corresponding 6-acetoxy-2-(4 acetoxyphenyl)benzo[b]thiophene, c) acylation of 6-acetoxy-2-(4-acetoxyphonyl)benzo[b]thiophene with 4-(2 piperidinoethoxy)benzoylchloride hydrochloride with aluminium trichloride in halogenated solvent to obtain 6-acetoxy-2-(4acetoxyphenyl)-3-[4-(2 piperidinoethoxy)benzoyl]-benzo[b]thiophene, d) hydrolysis of 6-acetoxy-2-(4-acetoxyphenyl)-3-[4-(2-piperidinoethoxy)benzoyll benzo[b]thiophene according to the following operating conditions: d1) treatment of 6-acetoxy-2-(4-acetoxyphonyl)-3-[4-(2-piperidinoethoxy)benzoyl]benzo[b]thiophene with alkaline hydroxide in alcohol solvent, d2) acidification of the product obtained in the preceding stage (d1) with a strong acid, to obtain the corresponding raloxifene salt with the strong acid, characterised in that the strong acid used in stage (d2) is concentrated hydrochloric acid.

### FIELD OF THE INVENTION

The present invention relates to a process for preparing raloxifene and in particular high purity raloxifene hydrochloride with high yields.

### STATE OF THE ART

Raloxifene and in particular the relating hydrochloride salt, characterised by the following formula (I): 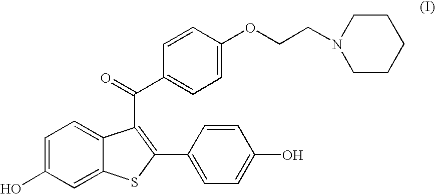
is an active principle used in the treatment of osteoporosis and was described for the first time in European patent application EP62503. In this prior patent various preparation methods are described which generally involve the following stages:
1) protection of the 2 hydroxylic functions of 6-hydroxy-2-(4-hydroxyphenyl)benzo[b]thiophene according to the following reaction scheme 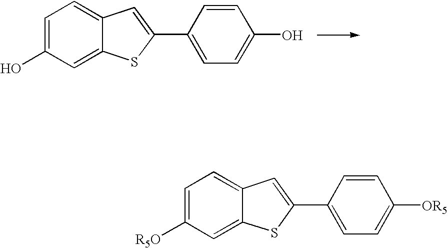
where R₅ is an alkyl, cycloalkyl or COR₆ acyl group, a SO₂R₆ sulfonyl group where R₆ is a primary or secondary C₁-C₄ alkyl, C₁-C₃ fluoro alkyl or C₁-C₄ alkoxyphenyl,
2) acylation of the compound protected with 4-(2-piperidinoethoxy)benzoyl halide according to the following synthesis scheme: 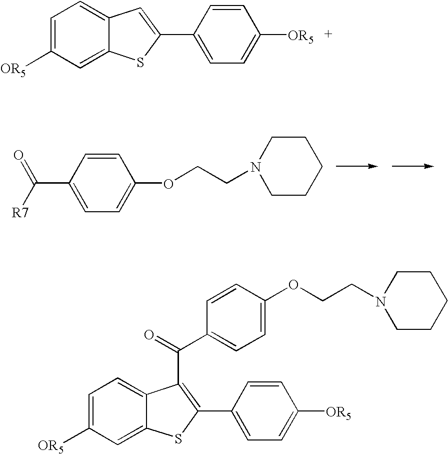
in which R₇ is a halogen atom,
3) deprotection or elimination of the OR₅ protective group.

As it results from the examples reported in EP62503, when the reaction is conducted using the acetyloxy group as OR₅ protective group, deprotection of this group is conducted first with sodium hydroxide in an alcoholic solution and subsequently with methanesulfonic acid. This type of hydrolysis however does not allow high purity raloxifene to be obtained, since, as indicated by example 6, the product to be purified must be passed through a chromatographic column. This type of treatment, however, only enables a yellow foam to be obtained, and, to arrive at a product of solid crystalline form, a further treatment with acetone is required. The crystallized product thus obtained consisting of raloxifene methanesulfonate must be further converted into the corresponding hydrochloride for pharmaceutical use.

The aforesaid process, requiring product passage through a chromatographic column, is not achievable at industrial level, proof of which being that in the same prior patent, instead of the aforesaid synthesis scheme, the one preferred is that in which the OR₅ protective group is an alkoxy, specifically a methoxy group, which for unblocking requires the use of aluminium trichloride and a thioderivative and preferably methanethiol, moreover in a quantity greatly in excess of the substrate on which the deprotection must be conducted, with considerable pollution problems, which evidently involves the use of considerable quantities of thioderivatives.

The processes described in EP62503 involve another inconvenience caused by the use of aluminium trichloride and, if proceeding to the scheme preferred by this prior patent, this Lewis acid must be used in substantial quantities, since it is used not only in stage (2) of acylation, but also in subsequent dealkylation. Aluminium trichloride as shown in the subsequent patent U.S. Pat. No. 5,629,425 produces a large quantity of aluminium-based by-products which are soluble in raloxifene processing solvents and are found therefore in the final product.

To overcome these problems, in the aforestated U.S. Pat. No. 5,629,425 boron trichloride or boron tribromide is used as Lewis acid, these being decidedly more expensive catalysts than aluminium trichloride.

The need was felt to provide a process which enabled raloxifene hydrochloride to be prepared with high yields and high purity and low aluminium content without using expensive catalysts.

### SUMMARY OF THE INVENTION

The applicant has surprisingly found a process capable of overcoming the drawbacks of known processes and which allows raloxifene and in particular raloxifene hydrochloride to be obtained with high purity and high yields.

This process comprises in particular the following stages:
a) demethylation of 6-methoxy-2-(4-methoxyphenyl)benzo[b]thiophene of formula (II) 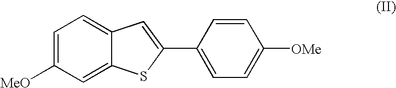
in pyridine hydrochloride to obtain 6-hydroxy-2-(4-hydroxyphenyl)benzo[b]thiophene of formula (III) 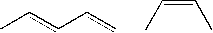
b) acetylation of 6-hydroxy-2-(4-hydroxyphenyl)benzo[b]thiophene with an acetylating agent to obtain the corresponding 6-acetoxy-2-(4-acetoxyphenyl)benzo[b]thiophene of formula (IV) 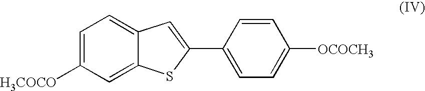
c) acylation of 6-acetoxy-2-(4-acetoxyphenyl)benzo[b]thiophene (IV) with 4-(2-piperidinoethoxy)benzoylchloride hydrochloride of formula (V) 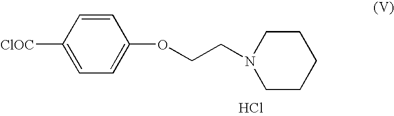
with aluminium trichloride in halogenated solvent to obtain 6-acetoxy-2-(4 acetoxyphenyl)-3-[4-(2-piperidinoethoxy)benzoyl]-benzo[b]thiophene of formula (VI) [symbol] 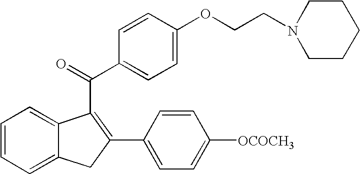
d) hydrolysis of 6-acetoxy-2-(4-acetoxyphenyl)-3-[4-(2-piperidinoethoxy)benzoyl]-benzo[b]thiophene, according to the following operative methods:
d1) treatment of 6-acetoxy-2-(4-acetoxyphenyl)-3-[4-(2-piperidinoethoxy)benzoyl]-benzo[b]thiophene with alkaline hydroxide in alcohol solvent,
d2) acidification of the product obtained in the previous stage (d1) with a strong acid, to obtain the corresponding raloxifene salt with strong acid, characterised in that the strong acid used in stage (d2) is concentrated hydrochloric acid.

In this respect, by conducting the hydrolysis of 6-acetoxy-2-(4-acetoxyphenyl)-3-[4-(2-piperidinoethoxy)benzoyl]-benzo[b]thiophene with sodium hydroxide and subsequently treating the product obtained with hydrochloric acid in place of methanesulfonic acid, raloxifene hydrochloride precipitates in crystalline form directly with a high purity equal to 98%, thus in contrast to the analogous process described in EP65203 conducted with methanesulfonic acid, without having to use purification processes such as passage through a chromatographic column, which are impractical from the industrial point of view. In addition the product derived from stage (d2) has a low aluminium content.

### DETAILED DESCRIPTION OF THE INVENTION

The 6-methoxy-2-(4-methoxyphenyl)benzo[b]thiophene of formula (II) used in stage (a) of the process of the present invention is prepared by reacting 3-methoxybenzene-thiol with α-bromo-4-methoxyacetophenone to obtain the corresponding α-(3-methoxyphenylthio)-4-methoxyacetophenone which is finally cyclizised to obtain the intermediate (II) with polyphosphoric acid, as in the following scheme.

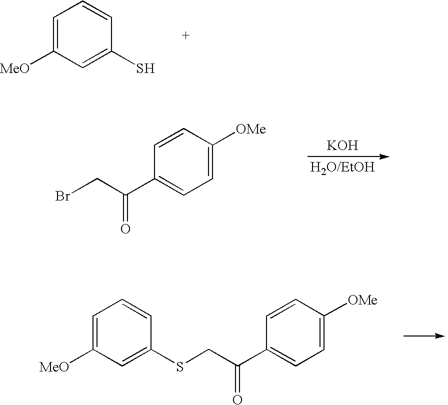

The pyridine hydrochloride used in stage (a) is preferably prepared in situ by adding concentrated hydrochloric acid to pyridine and distilling off all the water to obtain a thick but stirrable residue. The applicant has also surprisingly found that if the demethylation reaction or stage (a) of the process of the present invention is conducted in the presence not only of pyridine hydrochloride but also of tributylamine, preferably in weight ratios with respect to 6-methoxy-2-(4-methoxyphenyl)benzo[b]thiophene (II) of between 0.5 and 2, it is possible to lower the reaction temperature which in prior art is conducted at 210° C., to decidedly lower temperatures, between 170 and 180° C.

According to a preferred embodiment of the process of the present invention, it is not necessary to isolate the 6-hydroxy-2-(4-hydroxyphenyl)benzo[b]thiophene (III) obtained in stage (a).

In stage (b) according to a preferred embodiment acetic anhydride is used as acetylating agent and a tertiary aliphatic amine, preferably triethylamine, is used as hydrogen ion acceptor. The solvent used in stage (a) is an aprotic polar solvent, ethyl acetate being particularly preferred.

The 4-(2-piperidinoethoxy)benzoylchloride hydrochloride of formula (V) used in stage (c) is preferably prepared in situ by a conventional type procedure by reacting 4-(2-piperidinoethoxy)-benzoic acid hydrochloride with thionyl chloride without isolating the reaction product. This reaction is preferably conducted in methylene chloride in the presence of pyridine as catalyst.

Stage (c) is preferably conducted in methylene chloride, according to a particularly preferred embodiment this stage being conducted in the following manner: 6-acetoxy-2-(4-acetoxyphenyl)benzo[b]thiophene is added to 4-(2-piperidinoethoxy)benzoylchloride hydrochloride of formula (V) prepared in situ while still in its reaction solvent methylene chloride, the mixture thus obtained being poured onto a mixture consisting of methylene chloride and aluminium trichloride.

According to a preferred embodiment of the process of the present invention, 6-acetoxy-2-(4-acetoxyphenyl)-3-[4-(2-piperidinoethoxy)benzoyl]-benzo[b]thiophene (VI) is not isolated but is used in crude form for the subsequent hydrolysis (d).

Stage (d1) is preferably conducted using methanol as the alcoholic solvent, with excess 30% sodium hydroxide.

Stage (d2) is preferably conducted directly on the reaction mixture derived from stage (d1) to which equal weight quantities of water and ethyl acetate are added and finally 37% concentrated hydrochloric acid.

A suspension is hence obtained, which is preferably washed with equal weight quantities of water and ethyl acetate.

By the process of the present invention raloxifene hydrochloride is obtained with high purity and high yields of about 65-70% calculated on the 6-methoxy-2-(4-methoxyphenyl)benzo[b]thiophene (II).

The applicant has also found that if raloxifene hydrochloride obtained by the process of the present invention is crystallised from an alcoholic solvent, preferably methanol, possibly in the presence of small quantities of HCl, it achieves a purity of greater than 99%.

Finally the applicant has also found that by conducting a further crystallization, again from an alcoholic solvent, preferably methanol, possibly in the presence of HCl, on the product derived from the first crystallisation, raloxifene hydrochloride can be obtained with a purity greater than 99.7%. In particular raloxifene hydrochloride obtained after the first and/or the second crystallisation contains the characteristic impurity consisting of raloxifene hydrochloride N-oxide in a quantity less than 0.05% and preferably less than 0.01%, this product also having an aluminium content less than 5 ppm.

The product thus obtained has a particle size distribution (after gentle grinding conducted with the aim of simply homogenising the product) such that D(0.9) is ≦100 μm and D(0.5)≧40 μm. By further sieving a raloxifene hydrochloride is obtained with the following particle size distribution: D(0.9) between 50 and 65 μm and D[4.3]≧20 μm.

Some illustrative but non-limiting examples of the preparation process for raloxifene hydrochloride of the present invention and its relative intermediates are given.

### EXAMPLE 1

### Preparation of 6-acetoxy-2-(4-acetoxyphenyl)benzo[b]thiophene (IV)

24 kg of pyridine (0.303 kmol) and 28.8 kg of 37% hydrochloric acid (0.292 kmol) are fed into a reactor. The reactor is placed under vacuum and all the water is distilled off until a thick but stirrable residue is obtained.

The residue is then redissolved in 6 kg of tributylamine and 6 kg of 6-methoxy-2-(4-methoxyphenyl)benzo[b]thiophene (0.022 kmol). The mixture is heated to 170-180° C. and is maintained at this temperature for some hours. It is then cooled to 50-60° C. and 24 kg of ethyl acetate and 60 kg of deionised water are fed into the reactor. The mixture is stirred for 15 minutes and the phases are separated. The solvent is distilled off from the organic phase under vacuum and the residue is redissolved with 24 kg of ethyl acetate and 5.3 kg of triethylamine (0.052 kmol). The mixture obtained is heated to 60-65° C. while being stirred and 8.9 kg of acetic anhydride (0.087 kmol) are added. The reaction mixture is stirred for 1 hour at the same temperature then is cooled to 25-30° C. and 24 kg of deionised water are added. The suspension is centrifuged, washed with 6 kg of deionised water and 6 kg of ethyl acetate.

The product is then dried at 50-60° C. and about 6.6 kg of dried product are obtained. The reaction yield is 91.1%.

### EXAMPLE 2

Preparation of Crude Raloxifene Hydrochloride.

PHASE A

42 kg of methylene chloride and 7.8 kg of 4-(2-piperidinoethoxy)-benzoic acid hydrochloride (0.027 kmol), 0.12 kg pyridine (0.0015 kmol) are fed into a reactor and heated under reflux and then 3.96 kg of thionyl chloride (0.033 kmol) are added. The mixture is stirred for 1 hour then about 20 litres of methylene chloride are distilled off. The mixture is cooled to 20-30° C. and 6 kg of 6-acetoxy-2-(4-acetoxyphenyl)benzo[b]thiophene (IV) (0.018 kmol) are added.

The mixture is stirred until is completely homogenised.

PHASE B

36 kg of methylene chloride and 16.8 kg of aluminium trichloride (0.126 kmol) are fed into a reactor.

While stirring, the chloromethylene suspension, comprised of phase A prepared as described above, is added at 15-30° C. The mixture is stirred for 1 hour then the entire reaction mixture is poured into a reactor containing 60 kg of ice.

The mixture is stirred at 15-30° C. then the suspension is centrifuged, washing with 3 kg of methylene chloride and 3 kg of deionised water.

The centrifuged mother liquors, containing the product, are fed into a reactor and the phases are separated. The organic phase is distilled off until obtaining an oily residue and 15 kg of methyl alcohol are added, stirred at 20-40° C. and, maintaining the same temperature, 9.1 kg of 30% sodium hydroxide (0.068 kmol) are poured in. The mixture is stirred for 1 hour and 30 kg of deionised water and 30 kg of ethyl acetate are added.

At the same temperature 7.2 kg of 37% hydrochloric acid (0.073 kmol) are then added. The suspension is centrifuged, washing with 6 kg of ethyl acetate and 6 kg of deionised water. At the end 6.6 kg of dried product with HPLC purity>98% and low aluminium content are obtained. The reaction yield calculated on the 6-acetoxy-2-(4-acetoxyphenyl)benzo[b]thiophene (IV) is equal to a yield of 70.4%.

### EXAMPLE 3

Crystallisation of Crude Raloxifene Hydrochloride (1^{st} Crystallisation of Crude Raloxifene Hydrochloride)

6 kg of deionised water, 6 kg of crude raloxifene hydrochloride prepared as described in example 2 and 107 kg of methyl alcohol are fed into a reactor. The reaction mixture is heated until a complete solution is obtained then 0.25 kg of decolourising carbon are added. It is stirred for 15 minutes and then the suspension is filtered. While maintaining the solution stirred, 67 kg of methyl alcohol are distilled off. The residue is cooled and 0.1 kg of 37% hydrochloric acid are added. The pH, which must not exceed 2, is checked and the reaction mixture is then stirred for 2 hours at 20-40° C. The suspension is centrifuged, washing with 6 kg of methyl alcohol. 4.5 kg of dried product are obtained with HPLC purity of >99% and a yield of 75%.

### EXAMPLE 4

Crystallisation of Crystalline Raloxifene (2^{nd} Crystallisation).

0.9 kg of deionised water, 81 kg of methanol and the entire amount of crystallised product as described in example 3 are fed into a reactor. While maintaining the reaction mixture under stirring it is heated under reflux and 36 kg of methyl alcohol are distilled off. It is then cooled to 20-40° C. and 0.08 kg of 37% hydrochloric acid are added. The suspension is centrifuged, washing with 4 kg of methyl alcohol. The product is dried at 70° C. 4 kg of raloxifene hydrochloride are obtained with HPLC purity>99.8%, reaction yield 89%, in particular the raloxifene hydrochloride N-oxide content is less than 0.01% and aluminium content is less than 5 ppm. In particular the raloxifene hydrochloride obtained after crystallisation contains the characteristic impurity consisting of raloxifene hydrochloride N-oxide in a quantity less than 0.05% and preferably less than 0.01%. The product thus obtained has a particle size distribution (after gentle grinding conducted with the aim of simply homogenising the product) such that D(0.9) is ≦100 μm and D (0.5)≧40 μm.

By further sieving a raloxifene hydrochloride is obtained with the following particle size distribution: D(0.9) between 50 and 65 μm and D[4.3]≧20 μm.

1-25. (canceled)

26. Process for preparing raloxifene hydrochloride of formula (I) 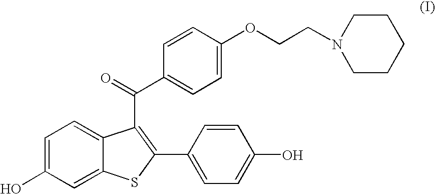
with a HPLC purity higher than 98% comprising the following stages:
a) demethylation of 6-methoxy-2-(4-methoxyphenyl)benzo[b]thiophene of formula (II) 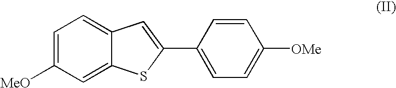
in pyridine hydrochloride to obtain 6-hydroxy-2-(4-hydroxyphenyl)benzo[b]thiophene of formula (III) 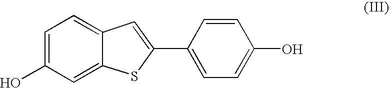
b) acetylation of 6-hydroxy-2-(4-hydroxyphenyl)benzo[b]thiophene with an acetylating agent to obtain the corresponding 6-acetoxy-2-(4-acetoxyphenyl)benzo[b]thiophene of formula (IV) 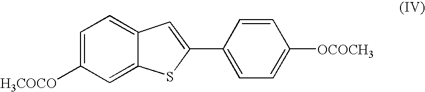
c) acylation of 6-acetoxy-2-(4-acetoxyphenyl)benzo[b]thiophene (IV) with 4-(2-piperidinoethoxy)benzoylchloride hydrochloride of formula (V) 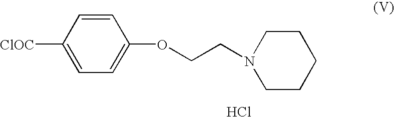
with aluminium chloride in halogenated solvent to obtain 6-acetoxy-2-(4-acetoxyphenyl)-3-[4-(2-piperidinoethoxy)benzoyl]-benzo[b]thiophene of formula (VI) 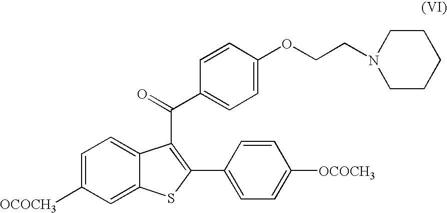
d) hydrolysis of 6-acetoxy-2-(4-acetoxyphenyl)-3-[4-(2-piperidinoethoxy)benzoyl]-benzo[b]thiophene, according to the following operative modalities:
d1) treatment of 6-acetoxy-2-(4-acetoxyphenyl)-3-[4-(2-piperidinoethoxy)benzoyl]-benzo[b]thiophene with alkaline hydroxide in alcohol solvent,
d2) acidification of the product obtained in the preceding stage (d1) with a strong acid, to obtain the corresponding raloxifene salt with the strong acid,
wherein:
stage (d1) is conducted using methanol as alcohol solvent and excess 30% sodium hydroxide,
the strong acid used in stage (d2) is concentrated hydrochloric acid and said stage (d2) is conducted directly on the reaction mixture derived from stage (d1) to which equal weight quantities of water and ethyl acetate and finally 37% concentrated hydrochloric acid are added.
the suspension obtained in stage (d2) is washed with equal weight quantities of water and ethyl acetate.

27. Process as claimed in claim 26, wherein the pyridine hydrochloride used in stage (a) is prepared in situ by adding concentrated hydrochloric acid to pyridine and distilling off all the water to obtain a thick but stirrable residue.

28. Process as claimed in claim 26, wherein the demethylation reaction or stage (a) of the process of the present invention is also conducted in the presence or tributylamine.

29. Process as claimed in claim 28, wherein tributylamine is used preferably in weight ratios with respect to 6-methoxy-2-(4-methoxyphenyl)benzo[b]thiophene (II) of between 0.5 and 2.

30. Process as claimed in claim 29, wherein stage (a) is conducted at a temperature between 170 and 180° C.

31. Process as claimed in claim 26, wherein acetic anhydride is used as acetylating agent in the presence of triethylamine in ethyl acetate.

32. Process as claimed in claim 26, wherein the 4-(2-piperidinoethoxy)benzoylchloride hydrochloride of formula (V) used in stage (c) is prepared in situ, by reacting 4-(2-piperidinoethoxy)benzoic acid hydrochloride with thionyl chloride in methylene chloride in the presence of pyridine, without isolating the reaction product.

33. Process as claimed in claim 26, wherein stage (c) is conducted in methylene chloride.

34. Process as claimed in claim 33, wherein stage (c) is conducted according to the following operative modalities: 6-acetoxy-2-(4-acetoxyphenyl)benzo[b]thiophene (IV) is added to non-isolated 4-(2-piperidinoethoxy)benzoylchloride hydrochloride of formula (V) and prepared in situ by reacting 4-(2-piperidinoethoxy) benzoic acid hydrochloride with thionyl chloride in methylene chloride in the presence of pyridine, without isolating the reaction product, and the aforesaid mixture is poured onto a mixture consisting of methylene chloride and aluminium trichloride.

35. Process as claimed in claim 26, wherein the 6-acetoxy-2-(4-acetoxyphenyl)benzo[b]thiophene (IV) is not isolated, but is used in the crude state in the subsequent reaction (d).

36. Process as claimed in claim 26, wherein raloxifene hydrochloride derived from stage (d2) is crystallised from an alcoholic solvent.

37. Process as claimed in claim 36, wherein said solvent is methanol possibly in the presence of HCl.

38. Process as claimed in claim 36, wherein raloxifene hydrochloride is obtained with a purity greater than 99%.

39. Process as claimed in claim 36, wherein a further crystallisation of raloxifene hydrochloride from alcohol solvent is conducted.

40. Process as claimed in claim 39, wherein said crystallisation is conducted in methanol possibly in the presence of HCl.

41. Raloxifene hydrochloride with a purity greater than 99.7% and containing aluminium in a quantity less than 5 ppm %.

42. Raloxifene hydrochloride as claimed in claim 41, containing raloxifene hydrochloride N-oxide in a quantity less than 0.05%.

43. Raloxifene hydrochloride as claimed in claim 42, wherein said impurity is contained in a quantity less than 0.01%.

44. Raloxifene hydrochloride as claimed in claim 43, having a D(0.9)≦100 μm and a D(0.5)≧40 μm.

45. Raloxifene hydrochloride as claimed in claim 44, having a D(0.9) between 50 and 65 μm and a D[4.3]≧20 μm.
